(19) United States
(12) Patent Application Publication
(10) Pub. No.: US 2017/0157027 A1
(43) Pub. Date: Jun. 8, 2017

(54) COMPOSITION FOR HAIR PACK AND A HAIR PACK COMPRISING THE SAME

(71) Applicant: Joo Won KIM, Hwaseong (KR)

(72) Inventor: Joo Won KIM, Hwaseong (KR)

(21) Appl. No.: 15/169,881

(22) Filed: Jun. 1, 2016

(30) Foreign Application Priority Data
Dec. 3, 2015 (KR) 10-2015-0171416

(51) Int. Cl. A61K 8/97; A61K 8/34; A61K 8/41; A61K 8/891; A61K 8/67; A45D 19/16; A61K 8/92; A61Q 5/12; A61Q 5/00; A61K 8/898; A61K 8/892; A61K 8/44; A61K 8/58

(52) U.S. Cl. CPC A61K 8/97; A61K 8/44; A61K 8/345; A61K 8/342; A61K 8/416; A61K 8/891; A61K 8/678; A61K 8/585; A61K 8/92; A61K 8/34; A61Q 5/12; A61Q 5/002; A61K 8/898; A61K 8/892; A45D 19/16; A61K 2800/242; A61K 2800/51; A61K 2800/594; A61K 2800/5922; A61K 2800/87; A45D 2019/0041

(57) ABSTRACT

The composition according to one embodiment of the present invention includes red pepper extract; and at least one extract selected from the group consisting of cabbage extract, broccoli extract, radish extract, and cauliflower extract, and can give thermal sensation, treat damaged hair, and improve hair condition. Furthermore, it is possible to care hair easily in house by using the hair pack to which the composition is applied.

### CROSS-REFERENCE TO RELATED APPLICATION

This application claims the benefit of Korean Patent Application No. 10-2015-0171416 filed on Dec. 3, 2015 in the Korean Intellectual Property Office, the disclosure of which is herein incorporated by reference in its entirety.

### TECHNICAL FIELD

The present invention relates to a composition for hair pack and a hair pack comprising the same.

### BACKGROUND OF THE INVENTION

Hair is damaged by chemical factors such as shampoo, perm, dyeing, and so on; environmental factors such as ultraviolet or polluted air; or physical factors such as brush or hair drier.

It is because hair, composed of protein, loses a part of protein or varies in characteristics and, as occasion demands, is weakened and easily split or cut by an external force.

Conventionally, in order to cure damaged hair like this or protect hair, it has been needed to be cared for at a hair salon or to be managed with products on sale.

However, it may be burden of cost and time to be cared for at a hair salon, and the products on sale have an inconvenience for customers to apply a hair nourishing agent to their hair and scalp by themselves. And, there was a problem of restriction on activity for the fixed time after the application of the hair nourishing agent.

In order to resolve such problem, a hair treatment pack materialized on a sheet has been reported (Patent Document 1).

However, the hair pack disclosed in Patent Document 1 has an inconvenience on the preparation and manufacturing processes, because the treatment solution in the pack is divided into the first agent and the second agent and it gives thermal sensation to users by a chemical reaction occurring when said two agents meet. And, there was an inconvenience on use because the thermal sensation cannot be felt if said two agents do not meet exactly when the hair pack is used.

### PRIOR ART DOCUMENTS

### Patent Documents

Korean Patent No. 10-1499478 (Date of Patent: Mar. 2, 2015)

### DETAILS OF THE INVENTION

### Objects of the Invention

The present invention provides a composition for hair pack that includes red pepper extract; and at least one extract selected from the group consisting of cabbage extract, broccoli extract, radish extract, and cauliflower extract, and can give thermal sensation, treat damaged hair, and improve hair condition, and a hair pack comprising the same.

### Means for Achieving the Object

Hereinafter, the composition for hair pack according to a concrete embodiment of the invention and the hair pack including the same are explained.

According to one embodiment of the invention, a composition for hair pack including red pepper extract; and at least one extract selected from the group consisting of cabbage extract, broccoli extract, radish extract, and cauliflower extract is provided.

The composition according to one embodiment is invented for resolving the problems of prior art, and it can provide a hair pack which gives thermal sensation when it is used to hair, and can cure damaged hair and improve hair condition, even though it does not include the first agent and the second agent separated.

The composition according to one embodiment includes a heating agent which gives thermal sensation to user when it is used to hair and cures damaged hair. The heating agent includes red pepper extract as an essential component; and at least one extract selected from the group consisting of cabbage extract, broccoli extract, radish extract, and cauliflower extract.

Said red pepper extract, cabbage extract, broccoli extract, radish extract, and cauliflower extract may be the extracts extracted from each material by various methods known in the art to which the present invention pertains.

For example, the extracts obtained by soaking each material in a suitable solvent and extracting active ingredients from each material may be used. Specifically, the extracts may be obtained by soaking each material in a suitable solvent, for example, an alcohol and the like, and filtering the same, after washing the same. The extracts may be used after passing a sterile filtration with necessity.

In the composition of one embodiment, the content of red pepper extract may be 0.1 to 5 wt % based on the total weight of the composition. If the content of red pepper extract is lower than above range, a curative effect for damaged hair is not sufficient because the thermal sensation is not sufficient, and if the content is higher than above range, there is a problem of that the thermal sensation is excessive and the composition is difficult to be used because it stimulates scalp excessively.

In the composition of one embodiment, the content of at least one extract selected from the group consisting of cabbage extract, broccoli extract, radish extract, and cauliflower extract may be 0.1 to 5 wt % based on the total weight of the composition. If the content of the extract is lower than above range, a curative effect for damaged hair is not sufficient because the thermal sensation is not sufficient, and if the content is higher than above range, there is a problem of that the thermal sensation is excessive and the composition is difficult to be used because it stimulates scalp excessively.

For optimizing the effect disclosed above, the composition according to one embodiment of the invention may include 0.10 to 2.00 wt % of cabbage extract, 0.10 to 2.00 wt % of broccoli extract, 0.10 to 2.00 wt % of radish extract, and 0.10 to 2.00 wt % of cauliflower extract, based on the total weight of the composition.

Meanwhile, the composition according to one embodiment of the invention may further include various other heating agents known in the related art as well as above extracts. For non-restrictive example, vanillyl butyl ether and so on may be used.

The total content of red pepper extract; at least one extract selected from the group consisting of cabbage extract, broccoli extract, radish extract, and cauliflower extract; and other heating agent besides above extracts may be adjusted to 0.2 to 15 wt % based on the total weight of the composition. In this range, it is possible to show a proper thermal sensation and to achieve a sufficient hair conditioning.

The composition according to one embodiment of the invention may further include various ingredients known in the art to which the present invention pertains, besides the heating agent. For example, the composition may further include a chelating agent, a moisturizer, an emulsifier, a hair conditioner, a preservative, a flavoring, or a mixture thereof.

Specifically, the chelating agent may include ethylenediamine, ethylenediamine tetraacetic acid, ethylenediamine triacetic acid, a salt thereof, or a mixture thereof. The content of the chelating agent may be 0.001 to 2 wt % based on the total weight of the composition.

The moisturizer may include one or more compounds selected from the group consisting of ethylene glycol, propylene glycol, butylene glycol, glycerin, dipropylene glycol, and an isomer thereof. The content of the moisturizer may be 1 to 30 wt % based on the total weight of the composition.

The emulsifier may include one or more alcohols selected from C₁₂-C₂₄ aliphatic alcohols. The content of the emulsifier may be 0.1 to 15 wt % based on the total weight of the composition.

The hair conditioner may include behentrimonium chloride, cetrimonium chloride, dimethicone, dimethiconol, amodimethicone, mineral oil, tocopheryl acetate, cyclotetrasiloxane, cyclopentasiloxane, cyclohexasiloxane, trideceth-12, polyquaternium, laureth-23, laureth-3, silicone quaternium-16, undeceth-11, butyloctanol, undeceth-5 or a mixture thereof. The content of the hair conditioner may be 5 to 50 wt % based on the total weight of the composition.

The preservative may include phenoxyethanol, ethylhexylglycerin, 1,2-hexanediol, or a mixture thereof. The content of the preservative may be 0.001 to 2 wt % based on the total weight of the composition.

The content of the flavoring may be 0.001 to 5 wt % based on the total weight of the composition. However, the ingredients of the composition according to one embodiment of the invention are not limited to the ingredients disclosed above and ingredients which have been commonly used in the art to which the present invention pertains may be used.

The composition according to one embodiment of the invention may further include the rest amount of a solvent in addition to above ingredients. The solvent may be an aqueous solvent such as water or an alcohol.

Meanwhile, according to another embodiment of the present invention, a hair pack, including a hair cap; and the composition of one embodiment impregnated in the hair cap, is provided.

The hair cap may be one of various hair caps known in the art to which the present invention pertains. For example, the hair cap may be prepared by preparing two sheets in which a nonwoven is laminated on a substrate composed of a synthetic resin, laminating two sheet so that nonwovens face each other, and sealing the edge of the same except an opening to which head of user is inserted. The nonwovens enable the composition for hair pack to be impregnated inside the hair cap prepared like this, and the outer substrate composed of a synthetic resin can prevent the composition for hair pack from leaking to the outside of the hair cap.

Particularly, the thermal sensation by the composition according to one embodiment of the invention can be maintained for a long time by using a heating material to the substrate or the nonwoven of the hair cap. The heating material may be an acrylate fiber such as heating fabric Exofil20 of Welcron Co. Ltd. Such heating fabric is expensive and it can effectively provide a hair pack representing excellent thermal sensation by using 10 to 30 wt % of the fabric to the total weight of the sheet.

The hair pack can represent the effect as much as steam is provided into the hair pack, because of the thermal sensation by the composition of one embodiment of the invention in addition to the heat generated from user's head. Accordingly, the hair pack may be called a steam hair pack. The hair pack shows excellent power of curing damaged hair and power of moisturizing hair, in comparison to existing hair pack products.

### Effects of the Invention

The composition according to one embodiment of the present invention includes red pepper extract; and at least one extract selected from the group consisting of cabbage extract, broccoli extract, radish extract, and cauliflower extract, and can give thermal sensation, treat damaged hair, and improve hair condition. Furthermore, it is possible to care hair easily in house by using the hair pack to which the composition is applied.

### BRIEF DESCRIPTION OF THE DRAWINGS

FIG. 1 represents SEM images of samples (1) and (3) prepared in Experimental Example 4.

### DETAILED DESCRIPTION OF THE EMBODIMENT

Hereinafter, functions and effects of the present invention will be explained in more detail with reference to the following examples. However, the following examples are only for illustrating the present invention, and the scope of the present invention is not limited to or by them.

### EXAMPLE 1

### Preparation of Composition for Hair Pack

Materials 1 to 4 and 8 disclosed in the following Table 1 were introduced into a main kiln in order and dissolved by heating the same to 80° C. Meanwhile, materials 5 to 7 and 9 to 14 were introduced into a separate assistant kiln and dissolved by heating the same to 80° C. And then, the mixture in the assistant kiln was introduced into the main kiln and emulsified. Subsequently, materials 15 to 18 were introduced to the main kiln in order and emulsified, and the main kiln was cooled to 45° C. And, materials 19 to 27 were introduced to the main kiln and emulsified again. And then, the main kiln was cooled to 30° C. and the obtained composition was defoamed under a vacuum condition and stored.

TABLE 1

| | | | Content |
|---|---|---|---|
| Specifications | No. | Name of material | [Unit: wt %] |
| Solvent | 1 | Purified water | 49.18 |
| Chelating agent | 2 | Disodium EDTA | 0.02 |
| Moisturizer | 3 | Glycerin | 10.00 |
| | 4 | Butylene glycol | 5.00 |
| Emulsifier | 5 | Cetearyl alcohol | 1.50 |
| | 6 | Stearyl alcohol | 1.50 |
| | 7 | Cetyl alcohol | 2.00 |
| Hair conditioner | 8 | Behentrimonium chloride | 8.50 |
| | 9 | Dimethicone | 7.00 |
| | 10 | Mineral oil | 2.00 |
| | 11 | Tocopheryl acetate | 0.10 |
| | 12 | Cyclopentasiloxane | 5.50 |
| | 13 | Cyclohexasiloxane | |
| | 14 | Dimethiconol | |
| | 15 | Amodimethicone | 5.00 |
| | 16 | Trideceth-12 | |
| | 17 | Cyclotetrasiloxane | |
| | 18 | Cetrimonium chloride | |
| Heating agent | 19 | Vanillyl butyl ether | 0.15 |
| | 20 | Red pepper extract | 0.50 |
| | 21 | Cabbage leaf extract | 0.30 |
| | 22 | Broccoli extract | 0.20 |
| | 23 | Radish extract | 0.20 |
| | 24 | Cauliflower extract | 0.30 |
| Preservative | 25 | Phenoxyethanol | 0.35 |
| | 26 | Ethylhexylglycerin | |
| Flavoring | 27 | Flavoring | 0.50 |

The extracts of red pepper, cabbage leaf, broccoli, radish, and cauliflower were obtained by the following method. First, after washing each material, it was soaked in ethanol and primarily filtered with 100 mesh filter and the residue was washed with ethanol. After mixing the obtained filtrate and the wash solution and storing the same at room temperature for 2 days, the mixture was secondarily filtered by using a filter of which the pore size is smaller than the filter used in the primary filtration. And then, the extract of each material was obtained by adding ethanol to the obtained filtrate and carrying out a sterile filtration.

### COMPARATIVE EXAMPLE 1

### Preparation of Composition for Hair Pack

The composition for hair pack was prepared by the same method as in Example 1, except that materials 21 to 24 of Example 1 were not used and the same amount of purified water as materials 21 to 24 was added thereto.

### EXPERIMENTAL EXAMPLE 1

### Evaluation on Thermal Sensation

<Sensory Evaluation>

In order to evaluate the thermal sensation of hair pack prepared according to one embodiment of the present invention, each of 20 testers used the hair packs of Example 1 and Comparative Example 1. The hair pack of Example 1 was prepared by applying the composition prepared in Example 1 to a hair cap, and the hair pack of Comparative Example 1 was prepared by applying the composition prepared in Comparative Example 1 to a hair cap.

Number of persons who answered “felt thermal sensation” was disclosed in ‘satisfied’ section, and number of persons who answered “didn't feel thermal sensation” was disclosed in ‘not satisfied’ section, after investigating whether they felt thermal sensation by using the hair pack or not.

TABLE 2

| | Example 1 | Comparative Example 1 |
|---|---|---|
| Satisfied | 18 | 10 |
| Not satisfied | 2 | 10 |

Though there was a difference between individuals as shown in Table 2, it was recognized that the hair pack of Example 1 showed more thermal sensation than the hair pack of Comparative Example 1.

<Measurement on Temperature Change>

The temperature change according to time after putting the hair pack of Example 1 and the hair pack of Comparative Example 1 on was measured with a thermo-graphic imaging camera (JEILMI, FLIR T420). And, the temperature changes as time goes on after putting the hair packs respectively impregnated with the compositions of Example 1 and Comparative Example 1 are listed in the following Table 3.

TABLE 3

| | 3 mins | 10 mins | 15 mins |
|---|---|---|---|
| Example 1 | 29.4° C. | 32.9° C. | 33.7° C. |
| Comparative | 26.4° C. | 28.7° C. | 31.2° C. |
| Example 1 | | | |

Referring to Table 3, it is recognized that the thermal sensation can be felt faster in the case of using the hair pack of Example 1 than the case of using the hair pack of Comparative Example 1 and the hair pack of the present invention can give the thermal sensation evenly to user's whole head.

### EXPERIMENTAL EXAMPLE 2

### Evaluation on Humidity

In order to check how much moisture can be applied to user's hair by using the hair pack according to one embodiment of the present invention, evaluation on humidity was carried out as follows.

Concretely, the humidity change between hair and hair cap or between hair and hair pack according to time of usage was measured in the cases of (1) putting a hair cap not impregnated with the composition on after shampooing and drying hair (indicated as ‘shampoo’ in Table 4), (2) putting a hair cap not impregnated with the composition on after shampooing, applying and washing a common treatment, and drying hair (indicated as ‘shampoo+treatment’ in Table 4), (3) putting a hair cap impregnated with the composition of Comparative Example 1 on after shampooing, applying and washing a common treatment, and drying hair (indicated as ‘shampoo+treatment+hair pack of Comparative Example 1’ in Table 4), (4) putting a hair cap impregnated with the composition of Example 1 on after shampooing, applying and washing a common treatment, and drying hair (indicated as ‘shampoo+treatment+hair pack of Example 1’ in Table 4). In the case of putting the hair pack impregnated with the composition of Example 1, the phenomenon of getting damp in the hair pack was observed.

The humidity change according to time of usage was measured by using a temperature and humidity measuring instrument (relative humidity: DT-171). The humidity change according to time of usage measured like this is shown in the following Table 4.

TABLE 4

| | 101 secs | 201 secs | 301 secs | 401 secs |
|---|---|---|---|---|
| (1) shampoo | 79.0 | 79.5 | 80.3 | 80.3 |
| (2) shampoo + | 77.9 | 79.7 | 80.5 | 80.7 |
| treatment | | | | |
| (3) shampoo + | 82.4 | 86.3 | 84.6 | 84.7 |
| treatment + | | | | |
| hair pack of | | | | |
| Comparative | | | | |
| Example | | | | |
| (4) shampoo + | 85.5 | 88.3 | 90.6 | 92.0 |
| treatment + | | | | |
| hair pack of | | | | |
| Example 1 | | | | |

(Unit: %)

Referring to Table 4, it is recognized that moisture can be sufficiently applied to hair by using the hair pack impregnated with the composition of Example 1.

### EXPERIMENTAL EXAMPLE 3

### Test on Water Loss

In order to recognize the moisture retention power of the hair pack according to one embodiment of the present invention, hair (1) was prepared by shampooing, hair (2) was prepared by shampooing, applying a common treatment, and washing, hair (3) was prepared by shampooing and using the hair pack impregnated with the composition of Example 1, and hair (4) was prepared by shampooing and using the hair pack impregnated with the composition of Comparative Example 1. The moisture rate (M₀) of hair samples (1) to (4) was measured, after drying the hair samples prepared in (1) to (4) by storing the same under the temperature of about 30° C. and the relative humidity of 60% for 6 hrs. And, the moisture rate (Mₜ) of hair samples (1) to (4) was measured again after drying samples (1) to (4) under the temperature of about 105° C. for 1 hr.

The water loss (%) was calculated by inserting the moisture rate of each sample measured in the following Equation 1.

Water Loss (%)=[(M₀−Mₜ)/M₀]*100 [Equation 1]

The water loss of each sample is shown in Table 5. Referring to Table 5, the water loss can be remarkably decreased in the case of using the hair pack according to one embodiment of the present invention.

TABLE 5

| | Sample 1 | Sample 2 | Sample 3 | Sample 4 |
|---|---|---|---|---|
| Water loss [%] | 17.2 | 15.6 | 10.4 | 12.5 |

### EXPERIMENTAL EXAMPLE 4

### Evaluation on Tensile Strength

In order to recognize the hair improvement power of the hair pack according to one embodiment of the present invention, hair (1) was prepared by shampooing, hair (2) was prepared by shampooing, applying a common treatment, and washing, hair (3) was prepared by shampooing and using the hair pack impregnated with the composition of Example 1, and hair (4) was prepared by shampooing and using the hair pack impregnated with the composition of Comparative Example 1. A strand of hair was collected among hair samples (1) to (4) and the tensile strength thereof was measured by using a universal testing machine (UTM-model 55651), after completely drying hair samples (1) to (4) with a hair drier.

The result of the tensile strength measurement to each sample is shown in the following Table 6.

TABLE 6

| | Sample 1 | Sample 2 | Sample 3 | Sample 4 |
|---|---|---|---|---|
| Tensile strength | 61.667 | 64.307 | 68.847 | 63.677 |
| [N/mm²] | | | | |

Referring to Table 6, sample (3) shows much higher tensile strength than samples (1), (2) and (4).

Comparing the condition of hair sample (1) and the condition of hair samples (3) and (4) with the naked eye, it is recognized that hair to which the hair pack according to one embodiment of the present invention was used is in good shape and has fine luster.

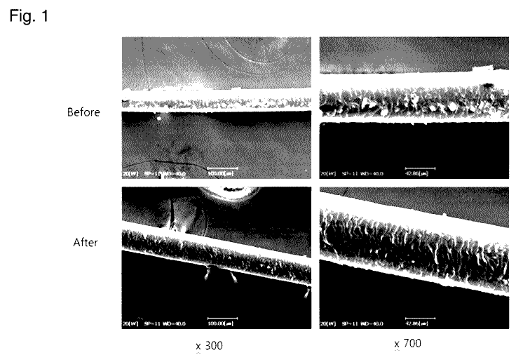

Particularly, referring to FIG. 1 that represents SEM photos of hair sample (1) and hair sample (3), it is recognized that the condition of hair is improved by using the hair pack according to one embodiment of the present invention.

1. A composition for hair pack, including:
red pepper extract; and
at least one extract selected from the group consisting of cabbage extract, broccoli extract, radish extract, and cauliflower extract.

2. The composition for hair pack according to claim 1, wherein the content of red pepper extract is 0.1 to 5 wt %, based on the total weight of the composition.

3. The composition for hair pack according to claim 1, wherein the content of at least one extract selected from the group consisting of cabbage extract, broccoli extract, radish extract, and cauliflower extract is 0.1 to 5 wt %, based on the total weight of the composition.

4. The composition for hair pack according to claim 1, including 0.10 to 2.00 wt % of cabbage extract, 0.10 to 2.00 wt % of broccoli extract, 0.10 to 2.00 wt % of radish extract, and 0.10 to 2.00 wt % of cauliflower extract, based on the total weight of the composition.

5. The composition for hair pack according to claim 1, further including a chelating agent, a moisturizer, an emulsifier, a hair conditioner, a preservative, a flavoring, or a mixture thereof.

6. The composition for hair pack according to claim 5, wherein the chelating agent includes ethylenediamine, ethylenediamine tetraacetic acid, ethylenediamine triacetic acid, a salt thereof, or a mixture thereof.

7. The position for hair pack according to claim 5, wherein the content of the chelating agent is 0.001 to 2 wt %, based on the total weight of the composition.

8. The position for hair pack according to claim 5, wherein the moisturizer includes one or more compounds selected from the group consisting of ethylene glycol, propylene glycol, butylene glycol, glycerin, dipropylene glycol, and an isomer thereof.

9. The position for hair pack according to claim 5, wherein the content of the moisturizer is 1 to 30 wt %, based on the total weight of the composition.

10. The position for hair pack according to claim 5, wherein the emulsifier includes one or more alcohols selected from C₁₂-C₂₄ aliphatic alcohols.

11. The position for hair pack according to claim 5, wherein the content of the emulsifier is 0.1 to 15 wt %, based on the total weight of the composition.

12. The position for hair pack according to claim 5, wherein the hair conditioner includes behentrimonium chloride, cetrimonium chloride, dimethicone, dimethiconol, amodimethicone, mineral oil, tocopheryl acetate, cyclotetrasiloxane, cyclopentasiloxane, cyclohexasiloxane, trideceth-12, polyquaternium, laureth-23, laureth-3, silicone quaternium-16, undeceth-11, butyloctanol, undeceth-5 or a mixture thereof.

13. The position for hair pack according to claim 5, wherein the content of the hair conditioner is 5 to 50 wt %, based on the total weight of the composition.

14. The position for hair pack according to claim 5, wherein the preservative includes phenoxyethanol, ethylhexylglycerin, 1,2-hexanediol, or a mixture thereof.

15. The position for hair pack according to claim 5, wherein the content of the preservative is 0.001 to 2 wt %, based on the total weight of the composition.

16. The position for hair pack according to claim 5, wherein the content of the flavoring is 0.001 to 5 wt % based on the total weight of the composition.

17. A hair pack, including:
a hair cap; and
the composition according to claim 1 impregnated in the hair cap.

18. The hair pack according to claim 17, wherein the hair cap includes an acrylate fiber which is a heating fabric.
